(19) United States
(12) Patent Application Publication
(10) Pub. No.: US 2017/0348239 A1
(43) Pub. Date: Dec. 7, 2017

(54) SOLID ORAL PHARMACEUTICAL COMPOSITION

(71) Applicant: Lupin Inc., Somerset, NJ (US)

(72) Inventors: James Garegnani, Somerset, NJ (US); Hiren Patel, Somerset, NJ (US)

(73) Assignee: Lupin Inc., Somerset, NJ (US)

(21) Appl. No.: 15/171,464

(22) Filed: Jun. 2, 2016

(51) Int. Cl. A61K 9/20; A61K 31/167; A61K 31/485; A61K 31/5415

(52) U.S. Cl. CPC A61K 9/2077; A61K 31/5415; A61K 31/167; A61K 31/485

(57) ABSTRACT

A solid oral pharmaceutical composition of promethazine, hydrocodone and acetaminophen, or a pharmaceutically acceptable salt thereof is provided. The composition comprises promethazine formulated for immediate release, both hydrocodone and acetaminophen formulated for controlled release, and at least one of the hydrocodone and acetaminophen formulated for immediate release.

### BACKGROUND OF THE INVENTION

### (a) Field of the Invention

The present invention is directed to a solid oral pharmaceutical composition comprising promethazine, hydrocodone, and acetaminophen or a pharmaceutically acceptable salt thereof. The invention is further directed to the use of said composition for treating or preventing pain, including migraine headache, and alleviating an adverse effect associated with administration of hydrocodone in individuals.

### (b) Description of the Related Art

Available pain medications may have adverse effects, such as nausea, vomiting, and skin rashes and sedation. As a result of such adverse effects, many subjects are unable to tolerate recommended dosages needed for effective pain relief because of adverse effects. Accordingly, there remains a need for effective therapeutics with reduced adverse effects.

Tablets containing a combination of hydrocodone bitartrate and acetaminophen is currently available in the US under the brand name Anexsia® from Mallinckrodt Pharmaceuticals.

U.S. Application Publication No. 2014/0288113 discloses a composition comprising an immediate release portion comprising hydrocodone, acetaminophen or a combination thereof, and an extended release portion comprising hydrocodone, acetaminophen or a combination thereof, wherein about 30% of the hydrocodone in the pharmaceutical composition is released in about 15 minutes and at least about 90% of the acetaminophen in the pharmaceutical composition is released in about 8 hours when measured in 900 ml of 0.1N HCl using a USP type II apparatus at a paddle speed of about 150 rpm and a constant temperature of 37° C.

U.S. Pat. No. 8,765,178 discloses a sustained release tablet with a specific geometric configuration for pain management. The tablet includes a first layer having a first active agent, where the first layer is disposed between two adjacent controlled release layers, at least one of the adjacent layers including at least one second active agent. The two adjacent layers are arranged such that they cover a portion of the first layer. The two adjacent layers may be separate layers or they may be joined into a single continuous layer, depending on the overall configuration and geometric design of the oral dosage form.

U.S. Pat. Nos. 8,124,126; 8,653,066 and 8,728,522 disclose a method of reducing or eliminating adverse effects associated with the use of analgesic agent, especially, an opioid analgesic agent. The method involves co-administration of promethazine, an antiemetic agent, with an opioid analgesic, hydrocodone and a non-opioid analgesic, acetaminophen. The patents also disclose multi-layered compositions comprising an immediate release layer of promethazine and a controlled release layer comprising hydrocodone and acetaminophen.

U.S. Application Publication Nos. 2015/0320685, 2015/0328209, 2015/0328210, 2015/0328211, 2015/0328213, 2014/0308349 and 2015/0290211 also disclose bilayer tablet formulations comprising an immediate layer of promethazine and a controlled release layer of hydrocodone and acetaminophen.

U.S. Application Publication Nos. 2015/0297525, 2015/0306040 and 2014/0134248 disclose a solid oral composition comprising a matrix formulated for immediate release of an antiemetic (e.g. promethazine) and another matrix formulated for controlled release of an opioid analgesic and a non-opioid analgesic.

U.S. Application Publication Nos. 2015/0290211, 2005/0232987, 2005/0266032 and 2005/0232986 disclose a pharmaceutical dosage form which comprises promethazine and at least one second drug, wherein the plasma half-life of the at least one second drug is shorter than a plasma half-life of promethazine by at least 3 hours. The dosage form provides a plasma concentration within a therapeutic range of the at least one second drug over a period which is coextensive with at least about 70% of a period over which the dosage form provides a plasma concentration within a therapeutic range of the first drug. The applications also disclose various compositions in which promethazine is formulated for immediate release and a second drug is formulated for controlled release.

A recently developed bilayer tablet composition by Charleston Labs (Florida, USA) contains an immediate release layer of promethazine and a controlled release layer comprising hydrocodone and acetaminophen. Clinical studies of this product for use in treating or preventing moderate to acute pain while preventing opioid induced nausea and vomiting (OINV) have been completed recently.

The prior art does not appear to disclose a pharmaceutical composition for pain management that includes one or more controlled release layers or portions of hydrocodone and acetaminophen and one or more immediate release layers or portions of promethazine, hydrocodone and acetaminophen. Such a pharmaceutical composition provides rapid onset of the therapeutic effects of the promethazine, hydrocodone and acetaminophen, and then provides long lasting therapeutic effects of the hydrocodone and acetaminophen. The prior art does not appear to disclose pharmaceutical compositions that provide such pain management.

Various options for pain management are available that are both immediate release and controlled release, and contain either a single drug or a combination of analgesics. While these combination products provide the benefits associated with combining two analgesics as described above, both immediate and controlled release, in itself, have a significant disadvantage. Immediate release combination products lack the advantages of controlled release products described previously. Controlled release combination products lack a significant benefit associated with immediate release products, namely, a rapid onset of analgesia that is extremely desirable for pain management. Because controlled release products retard the rate of drug release to sustain the drug effect over prolonged period, release of drug is slow resulting in a significant time before effective analgesic drug concentration is attained in the bloodstream.

There still exists a need for effective management of pain with a combination composition comprising promethazine, hydrocodone and acetaminophen that reduces or eliminates side effects associated with use of hydrocodone, provides instant and/or prolonged relief from pain immediately after administration of such composition and that combines the desirable features of immediate release and controlled release in such combination pain products.

### SUMMARY OF THE INVENTION

The present invention provides the following aspects, subject-matters and preferred embodiments, which respectively taken alone or in combination, further contribute to solving the object of the present invention.

As described in further detail below, advantageously the solid oral pharmaceutical composition comprises two portions of hydrocodone or acetaminophen or both drugs. One portion is formulated for controlled release and the other portion is formulated for immediate release. The immediate release portion of the two actives in the formulation may achieve desired therapeutic concentration of these actives instantly, or substantially instantly, upon administration.

In another aspect, the solid pharmaceutical composition includes one or more controlled release layers or portions of hydrocodone and acetaminophen and one or more immediate release layers or portions of promethazine, hydrocodone and acetaminophen.

In another aspect, the solid pharmaceutical composition consists of one or more controlled release layers or portions of hydrocodone and acetaminophen and one or more immediate release layers or portions of promethazine, hydrocodone and acetaminophen.

In another aspect, the solid pharmaceutical composition consists essentially of one or more controlled release layers or portions of hydrocodone and acetaminophen to provide the therapeutic effects of these two active compounds for an extended period and one or more immediate release layers or portions of promethazine, hydrocodone and acetaminophen to provide the therapeutic effects of these three active compounds initially and rapidly upon administration.

The controlled release layers or portions may be devoid of any promethazine and the presence of the promethazine is limited to the immediate release layers or portions.

Such a pharmaceutical composition provides rapid onset of the therapeutic effects of the promethazine, hydrocodone and acetaminophen, and then provides long lasting therapeutic effects of the hydrocodone and acetaminophen.

In one aspect, the present invention provides a solid oral pharmaceutical composition comprising:
(a) promethazine or a pharmaceutically acceptable salt thereof formulated for immediate release,
(b) hydrocodone or a pharmaceutically acceptable salt thereof formulated for controlled release,
(c) acetaminophen or a pharmaceutically acceptable salt thereof formulated for controlled release; and
(d) at least one of hydrocodone and acetaminophen or a pharmaceutically acceptable salt thereof formulated for immediate release.

In a further aspect, there is provided a solid oral pharmaceutical composition comprising promethazine, hydrocodone and acetaminophen, or a pharmaceutically acceptable salt thereof, wherein the composition comprises:
(a) a plurality of inert spheres coated with one or more layers of hydrocodone, or a pharmaceutically acceptable salt thereof, and one or more outer layers of release rate controlling excipients,
(b) a plurality of granules comprising acetaminophen, or a pharmaceutically acceptable salt thereof, and one or more release rate controlling excipients; and
(c) an extra-granular admixture comprising promethazine, hydrocodone, acetaminophen, or a pharmaceutically acceptable salt thereof, and one or more pharmaceutically acceptable excipients.

The inert spheres in the composition may be selected from sugar spheres, non-pareil seeds, microcrystalline cellulose beads, or mixtures thereof.

The hydrocodone layer and release rate controlling excipient layer in the composition may be separated by a barrier layer.

In a further aspect, more than 60% of the total amount of hydrocodone and acetaminophen or a pharmaceutically acceptable salt thereof are formulated for controlled release.

In a further aspect, up to 40% of the total amount of each of hydrocodone and acetaminophen, or a pharmaceutically acceptable salt thereof in the composition are formulated for immediate release.

In a further aspect, about 90% of the total amount of hydrocodone and acetaminophen or a pharmaceutically acceptable salt thereof are formulated for controlled release.

In a further aspect, the composition comprises about 6 mg to about 15 mg hydrocodone or a pharmaceutically acceptable salt thereof. In a further aspect, the composition comprises about 7.5 mg hydrocodone or a pharmaceutically acceptable salt thereof.

In a further aspect, the composition comprises about 300 mg to 500 mg acetaminophen or a pharmaceutically acceptable salt thereof. In a further aspect, the composition comprises about 325 mg acetaminophen or a pharmaceutically acceptable salt thereof.

In a further aspect, the composition comprises about 10 mg to 15 mg promethazine or a pharmaceutically acceptable salt thereof. In a further aspect, the composition comprises about 12.5 mg promethazine or a pharmaceutically acceptable salt thereof.

In a further aspect, the composition comprises the promethazine or a pharmaceutically acceptable salt thereof, the acetaminophen or a pharmaceutically acceptable salt thereof, and the hydrocodone or a pharmaceutically acceptable salt thereof in a mass ratio of about (1 to 2):(40 to 45):(1 to 2).

In a further aspect, the composition, on contact with a dissolution fluid as measured by a USP Apparatus 2 (Paddle Apparatus) exhibits at least one of the following profiles:
(a) up to 25% of the hydrocodone, or a pharmaceutically acceptable salt thereof is released in up to 4 minutes;
(b) up to 70% of the hydrocodone, or a pharmaceutically acceptable salt thereof is released in up to 12 minutes;
(c) up to 85% of the hydrocodone, or a pharmaceutically acceptable salt thereof is released in up to 60 minutes; or
(d) up to 90% of the promethazine, or a pharmaceutically acceptable salt thereof is released in up to 12 minutes.

In a further aspect, release of promethazine, hydrocodone, acetaminophen, or a pharmaceutically acceptable salt thereof from the composition commences simultaneously.

In a further aspect, the present invention provides a tablet comprising
(a) promethazine or a pharmaceutically acceptable salt thereof formulated for immediate release,
(b) a plurality of components comprising hydrocodone or a pharmaceutically acceptable salt thereof formulated for controlled release,
(c) a plurality of components comprising acetaminophen or a pharmaceutically acceptable salt thereof formulated for controlled release; and
(d) at least one of hydrocodone, acetaminophen or a pharmaceutically acceptable salt thereof formulated for immediate release.

In another aspect, the components formulated for controlled release are in the form of coated and/or matrix granules, mini-tablets, beads, pellets, or mixtures thereof.

In another aspect, the components formulated for immediate release are in the form of a powder blend or as a coating over the core (e.g. tablet).

In a further aspect, the components comprising hydrocodone or acetaminophen or a pharmaceutically acceptable salt thereof formulated for controlled release comprise:
(a) a core comprising hydrocodone and/or acetaminophen or a pharmaceutically acceptable salt thereof, optionally with one or more release rate controlling excipients; and
(b) a coating over the core comprising one or more release rate controlling excipients.

In a further aspect, the components comprising hydrocodone or acetaminophen or a pharmaceutically acceptable salt thereof formulated for controlled release comprise a core comprising a matrix of hydrocodone and/or acetaminophen or a pharmaceutically acceptable salt thereof, and one or more release rate controlling excipients.

In another aspect, the present invention provides a method for treating or preventing pain comprising administering to a subject in need thereof the solid oral pharmaceutical composition as substantially described throughout the specification.

In another aspect, the present invention provides a method for alleviating an adverse effect associated with administration of hydrocodone comprising administering to a subject in need thereof the solid oral pharmaceutical composition as substantially described throughout the specification.

In another aspect, the present invention provides a method for treating moderate to severe acute pain while preventing opioid (hydrocodone) induced nausea and vomiting comprising administering to a subject in need thereof the solid oral pharmaceutical composition as substantially described throughout the specification.

Still other aspects and advantages of the invention will be apparent from the following detailed description of the invention.

### DETAILED DESCRIPTION OF THE INVENTION

The invention is generally directed to compositions comprising multiple pharmaceutically active agents that are useful as therapeutics that alleviate, abate or eliminate one or more conditions in a subject in need thereof and that provide instant and effective relief from pain, as further described herein below.

The solid oral pharmaceutical composition of the invention comprises promethazine, hydrocodone, acetaminophen or a pharmaceutically acceptable salt thereof, in which promethazine is formulated for immediate release and hydrocodone and acetaminophen are formulated to provide immediate release as well as controlled release. In the composition, both hydrocodone and acetaminophen are formulated for controlled release while either hydrocodone or acetaminophen or both drugs are also separately formulated for immediate release.

The inventors have found that by initiating the simultaneous release of all three drugs upon administration, such composition can achieve therapeutic concentration of opioid and/or non-opioid drug immediately upon administration as compared to the known compositions. As a result of this characteristic, the composition of invention may produce instant and/or prolonged relief from pain.

The term “about” means the referenced numeric indication plus or minus 10% of that referenced numeric indication.

The term “matrix” as used herein throughout the specification denotes that the drugs and release rate controlling excipients, wherever applicable, are dispersed with one or more pharmaceutically acceptable excipients either homogeneously or heterogeneously. For example, in the homogeneous matrix system the drugs and optional release rate controlling excipients are distributed uniformly over the entire core, while in the heterogeneous matrix system the drugs and optionally release rate controlling excipients are non-uniformly distributed over the entire core.

The term “controlled-release” refers to the release of at least one pharmaceutically active agent from a dosage form at a particular desired point in time after the dosage form is administered to a subject. Generally, controlled-release includes sustained but otherwise complete release. A sudden and total release in the stomach at a desired and appointed time or a release in the intestine, such as through the use of an enteric coating, are both considered controlled-release. Controlled-release can occur at a predetermined time or in a predetermined place within the digestive tract. It is not meant to be a passive, uncontrolled process as in swallowing a normal tablet.

A control release dosage form begins its release and continues that release over an extended period of time. Release can occur beginning almost immediately or can be sustained. Release can be constant, can increase or decrease over time, can be pulsed, can be continuous or intermittent, and the like. Generally, however, the release of at least one pharmaceutically active agent from a controlled-release dosage form will exceed the amount of time of release of the drug taken as a normal, passive release tablet. Controlled-release in accordance with the compositions and methods described herein generally means that the release occurs for a period of six hours or more, such as 12 hours or more.

In one embodiment, controlled-release refers to delayed release of an agent from a composition or dosage form in which the agent is released according to a desired profile in which the release occurs after a period of time.

In one embodiment, the composition comprises an effective amount of hydrocodone or a pharmaceutically acceptable salt thereof, an effective amount of acetaminophen or a pharmaceutically acceptable salt thereof, and an effective amount of promethazine or a pharmaceutically acceptable salt thereof.

In one embodiment, about 70% to about 80% of a pharmaceutically active agent is capable of achieving dissolution from the immediate release part at about 10 minutes following oral administration or following contact with a dissolution fluid.

In another embodiment, about 100% of a pharmaceutically active agent is capable of achieving dissolution from the immediate release part at about 40 minutes following oral administration or following contact with a dissolution fluid.

In another embodiment, about 30% to about 40% of a pharmaceutically active agent is capable of achieving dissolution from the controlled release part at about 10 minutes following oral administration or following contact with a dissolution fluid.

In another embodiment, about 70% or less of a pharmaceutically active agent is capable of achieving dissolution from the controlled release part at about 60 minutes following oral administration or following contact with a dissolution fluid.

In another embodiment, 90% or less of the promethazine dissolves in the stomach of a subject in about 12 minutes following oral administration or following contact with a dissolution fluid.

In another embodiment, up to about 25% of the hydrocodone dissolves in the stomach of a subject in about 4 minutes following oral administration or following contact with a dissolution fluid.

In another embodiment, up to about 70% of the hydrocodone dissolves in the stomach of a subject in about 12 minutes following oral administration or following contact with a dissolution fluid.

In another embodiment, up to about 85% of the hydrocodone dissolves in the stomach of a subject in about 60 minutes following oral administration or following contact with a dissolution fluid.

In one embodiment, promethazine that reduces or eliminates an adverse effect associated with administration of hydrocodone or acetaminophen is released simultaneously to hydrocodone or acetaminophen in composition of the invention.

In one embodiment, a composition comprises hydrocodone, acetaminophen and promethazine, wherein the composition is capable of providing an effective plasma concentration of all three drugs simultaneously post oral administration.

In an embodiment, release of promethazine, hydrocodone, acetaminophen, or a pharmaceutically acceptable salt thereof from the composition commences simultaneously.

In one embodiment, the composition is capable of providing an effective plasma concentration of the promethazine in about 1 minute to about 20 minutes after administration to a subject.

In some embodiments, a dosage form of the invention provides an effective plasma concentration of hydrocodone or acetaminophen at from about 1 minutes to about 24 hours after administration, such as about 1 minute, 3 minutes, 5 minutes, 10 minutes, 15 minutes, 20 minutes, 30 minutes, 40 minutes, 50 minutes, 1 hr, 1.2 hrs, 1.4 hrs, 1.6 hrs, 1.8 hrs, 2 hrs, 2.2 hrs, 2.4 hrs, 2.6 hrs, 2.8 hrs, 3 hrs, 3.2 hrs, 3.4 hrs, 3.6 hrs, 3.8 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, or 24 hrs following administration.

The invention further provides methods and compositions formulated for oral delivery to a subject in need.

In one embodiment, compositions are provided in modified release dosage forms (such as immediate release, controlled release or both), which comprise an effective amount of hydrocodone or a salt thereof, acetaminophen or a salt thereof, and promethazine or a salt thereof; and one or more release rate controlling excipients as described herein. Preferable modified release dosage vehicles include, but are not limited to, hydrophilic or hydrophobic matrix devices, water-soluble separating layer coatings, enteric coatings, osmotic devices, multi-particulate devices, and combinations thereof. The compositions may also comprise non-release controlling excipients.

In another embodiment, compositions can be provided in a dosage form that has at least one component that can facilitate the immediate release of an active agent, and at least one component that can facilitate the controlled release of an active agent. In a further embodiment the dosage form can be capable of giving a discontinuous release of the compound in the form of at least two consecutive pulses separated in time from 0.1 up to 24 hours. The compositions can comprise one or more release controlling and non-release controlling excipients, such as those excipients suitable for a disruptable semi-permeable membrane and for use as swellable substances.

The compositions provided herein can be in unit-dosage forms or multiple-dosage forms. Unit-dosage forms, as used herein, refer to physically discrete units suitable for administration to human subjects and packaged individually. Each unit-dose can contain a predetermined quantity of an active ingredient(s) sufficient to produce the desired therapeutic effect, in association with the required pharmaceutical carriers or excipients. Examples of unit-dosage forms include, but are not limited to tablets and capsules. Unit-dosage forms may be administered in fractions or multiples thereof. A multiple-dosage form is a plurality of identical unit-dosage forms packaged in a single container, which can be administered in segregated unit-dosage form. Examples of multiple-dosage forms include, but are not limited to, bottles of tablets or capsules.

In an embodiment, the solid oral pharmaceutical composition is in the form of a tablet comprising:
(a) promethazine or a pharmaceutically acceptable salt thereof formulated for immediate release;
(b) a plurality of components comprising hydrocodone or a pharmaceutically acceptable salt thereof formulated for controlled release;
(c) a plurality of components comprising acetaminophen or a pharmaceutically acceptable salt thereof formulated for controlled release; and
(d) at least one of hydrocodone, acetaminophen or a pharmaceutically acceptable salt thereof formulated for immediate release.

In a preferred embodiment, the solid oral pharmaceutical composition comprises:
(a) a plurality of inert spheres coated with one or more layers of hydrocodone, or a pharmaceutically acceptable salt thereof, and one or more outer layers of release rate controlling excipients;
(b) a plurality of granules comprising acetaminophen, or a pharmaceutically acceptable salt thereof, and one or more release rate controlling excipients; and
(c) an extra-granular admixture comprising promethazine, hydrocodone, acetaminophen, or a pharmaceutically acceptable salt thereof, and one or more pharmaceutically acceptable excipient.

The inert spheres in the composition may be selected from sugar spheres, non-pareil seeds, microcrystalline cellulose beads, or mixtures thereof.

The hydrocodone layer and the release rate controlling excipient layer in the composition may be separated by a barrier layer.

In one embodiment, the components formulated for controlled release are in the form of coated or matrix granules, mini-tablets, beads, pellets, or mixtures thereof.

In a further embodiment, the components formulated for immediate release are in the form of powder blend or as a coating over the core (e.g. tablet).

In a further embodiment, the components comprising hydrocodone or acetaminophen or a pharmaceutically acceptable salt thereof formulated for controlled release comprise:
(a) a core comprising hydrocodone or acetaminophen or a pharmaceutically acceptable salt thereof, optionally with one or more release rate controlling excipients; and
(b) a coating over the core comprising one or more release rate controlling excipients.

In a further embodiment, the components comprising hydrocodone or acetaminophen or a pharmaceutically acceptable salt thereof formulated for controlled release comprises a core comprising hydrocodone or acetaminophen or a pharmaceutically acceptable salt thereof and with one or more release rate controlling excipients.

In one embodiment, the tablet comprises:
(A) a core which comprises of:
   (a) a plurality of components comprising hydrocodone or a pharmaceutically acceptable salt thereof formulated for controlled release; and
   (b) a plurality of components comprising acetaminophen or a pharmaceutically acceptable salt thereof formulated for controlled release;
(B) a layer over the core comprising promethazine, hydrocodone, acetaminophen, or a pharmaceutically acceptable salt thereof formulated for immediate release.

The dosage forms described herein can be manufactured using processes that are well known to those of skill in the art. For example, for the manufacture of tablets, the agents can be dispersed uniformly in one or more excipients, for example, using high shear granulation, low shear granulation, fluid bed granulation, or by blending for direct compression.

Controlled release of the drugs from the pharmaceutical composition is achieved by using a suitable release rate controlling excipient of hydrophilic, lipophilic or inert character or a combination of several different release rate controlling excipients providing controlled release of the drugs.

Suitable release rate controlling excipients, by way of example and without limitation, include the following. For example, in an embodiment, the release rate controlling excipient is selected from the group consisting of hydrophilic agents, lipophilic agents and inert agents. The hydrophilic agents are selected from the group of pharmaceutical excipients which generate a gel in contact with water, including cellulose derivatives such as hydroxypropyl methyl cellulose, hydroxyethyl cellulose, hydroxypropyl cellulose, methyl cellulose and the like; noncellulose polysaccharides such as galactomannanes, guar gum, carob gum, gum arabicum, alginates, pectins, and the like; polyvinylpyrrolidone; polyvinylacetate polymers and copolymers; acrylic acid polymers and copolymers, polyethylene oxide and mixtures thereof. The lipophilic agents are selected from the group consisting of waxes such as white wax, bees wax, carnauba wax and the like; fatty acids and alcohols such as stearic acid, palmitic acid, lauric acid and the like, and cetyl alcohol, cetostearyl alcohol, stearyl alcohol and the like; fatty acids esters such as monostearates of propylene glycol and fatty acid esters of sucrose, sucrose distearate and the like; and glycerides such as mono-, di- or triglycerides, e.g. palmitin, stearin, behenic, laurin, myristin, hydrogenated vegetable, castor, cottonseed oils, glyceril behenate and the like; and mixtures thereof. The inert agents are selected from the group consisting of thermoplastic polymers, which are insoluble and indigestible in the gastrointestinal fluids, such as polyvinyl chloride, polyethylene, vinyl acetate/vinyl chloride copolymers, polymethylmethacrylates, polyamides, silicones, ethyl cellulose, polystyrene, and mixtures thereof.

In an embodiment, controlled release formulations can comprise one or more combinations of excipients that slow the release of the agents by coating or temporarily bonding or decreasing their solubility of the active agents. Examples of these excipients include ethyl cellulose, cellulose ethers such as hydroxypropyl methyl cellulose (e.g., Methocel K4M) or silicified microcrystalline cellulose, polyvinylacetate-based excipients such as, e.g., Kollidon SR, and polymers and copolymers based on methacrylates and methacrylic acid such as, e.g., Eudragit NE 30D.

The present compositions can further comprise suitable additives, including, but not limited to, diluents, binders, surfactants, lubricants, glidants, coating materials, plasticizers, coloring agents, flavoring agents, or pharmaceutically inert materials.

Examples of diluents include, for example, cellulose; cellulose derivatives such as microcrystalline cellulose and the like; starch; starch derivatives such as corn starch, cyclodextrin and the like; dry starch; hydrolyzed starches, sugar; sugar alcohol such as lactose, mannitol and the like; inorganic diluents such as dried aluminum hydroxide gel, precipitated calcium carbonate, magnesium aluminometasilicate, dibasic calcium phosphate, sodium chloride, silicon dioxide, titanium oxide, dicalcium phosphate dihydrate, calcium sulfate, calcium carbonate, alumina and kaolin and the like.

Examples of binders include, for example, starch (including corn starch and pregelatinized starch), gelatin, sugars (e.g., glucose, dextrose, sucrose, lactose and sorbitol), celluloses (hydroxypropylcellulose, methylcellulose, hydroxypropyl methyl cellulose), polyethylene glycol, waxes, dextrin, natural and synthetic gums, e.g., acacia, pullulane, tragacanth, sodium alginate, and synthetic polymers such as polymethacrylates and polyvinylpyrrolidone

Examples of surfactants include, for example, sucrose esters of fatty acids, polyoxyl stearate, polyoxyethylene hydrogenated castor oil, polyoxyethylene polyoxypropylene glycol, sorbitan sesquioleate, sorbitan trioleate, sorbitan monostearate, sorbitan monopalmitate, sorbitan monolaurate, polysorbate, glyceryl monostearate, sodium lauryl sulfate, lauromacrogol and the like.

Examples of lubricants include, for example, stearic acid, calcium stearate, magnesium stearate, talc, glyceryl behenate, and polyethylene glycol.

Examples of disintegrants include, for example, starches, alginic acid, crosslinked polymers such as, e.g., crosslinked polyvinylpyrrolidone, croscarmellose sodium, potassium or sodium starch glycolate, clays, celluloses, starches, gums and the like.

Examples of glidants include, for example, silicon dioxide, talc, dried aluminum hydroxide gel, magnesium silicate and the like.

Examples of coating materials include, for example, hydroxypropyl methyl cellulose 2910, aminoalkyl methacrylate copolymer E, polyvinylacetal diethylaminoacetate, macrogol 6000, titanium oxide and the like. Examples of plasticizers include, for example, triethyl citrate, triacetin, macrogol 6000 and the like.

If desired, the composition can also comprise nontoxic, pharmaceutically acceptable auxiliary substances such as pH buffering agents, preservatives, e.g., antioxidants, wetting or emulsifying agents, solubilizing agents, coating agents, and the like.

In various embodiments, the composition is in the form of any oral dosage form disclosed herein, including but not limited to a pill, tablet, or capsule. In one embodiment, the composition is in the form of a single layer tablet having an immediate release part and a controlled release part, wherein one or more pharmaceutically active agents are present in the immediate release part and one or more pharmaceutically active agents are present in the controlled release part. In another embodiment, the immediate release part comprises promethazine, and the controlled-release portion comprises hydrocodone and acetaminophen. In a further embodiment, hydrocodone and acetaminophen are present in both the immediate-release and controlled release parts.

In one embodiment, the compositions comprise: hydrocodone, or a pharmaceutically acceptable salt thereof, in a dosage range of from about 1.0 mg to about 200 mg; acetaminophen or a pharmaceutically acceptable salt thereof in a dosage range of from about 200 mg to about 1000 mg; and promethazine or a pharmaceutically acceptable salt thereof in a dosage range of from about 0.5 mg to about 100 mg.

The pharmaceutical composition may be configured in various shapes and sizes for ease of administration to a patient. Manufacture of pharmaceutical compositions configured in tablets comprises steps known in the art. For example, tablets may be prepared through wet-granulation, dry-granulation or direct compression.

In some embodiments the composition further comprises an effective amount of an opioid antagonist agent or abuse deterrent agent.

The composition may contain one or more additional drugs that reduce or eliminate an adverse effect, preferably an antiemetic agent or antihistamine, including, dolasetron, granisetron, ondansetron, tropisetron, palonosetron, domperidone, droperidol, haloperidol, chlorpromazine, prochloperazine, metoclopramide, alizapride, cyclizine, diphenhydramine, dimenhydrinate, meclizine, hydroxyzine, cannabis, dronabinol, nabilone, midazolam, lorazepam, hyoscine, dexamethasone, trimethobenzamide, emetrol, and propofol or a pharmaceutically acceptable salt thereof.

In a further embodiment, the composition may contain one or more additional opioid analgesic agents, including oxycodone, morphine, codeine, narcotine, papaverine, narceine, thebaine, or a pharmaceutically acceptable salt thereof and non-opioid analgesic agents, including, ibuprofen, naproxen or flubiprofen, or a pharmaceutically acceptable salt thereof.

In one embodiment, a method is provided for reducing or eliminating an adverse effect of an opioid analgesic, hydrocodone, comprising administering to a subject in need thereof a composition comprising an effective amount of each of hydrocodone, or a pharmaceutically acceptable salt thereof, acetaminophen, or a pharmaceutically acceptable salt thereof, and promethazine, or a pharmaceutically acceptable salt thereof.

In one embodiment, a method is provided for treating or preventing pain, comprising administering to a subject in need thereof an effective amount of a composition comprising an effective amount of each of hydrocodone, or a pharmaceutically acceptable salt thereof, acetaminophen, or a pharmaceutically acceptable salt thereof, and promethazine, or a pharmaceutically acceptable salt thereof. Preferably, the pain is due to headache.

The methods allow for use of hydrocodone in populations at risk of adverse effect such as nausea, vomiting, other gastric upsets, skin rashes, allergic reactions such as swelling, difficulty breathing, closing of throat, abdominal pain, unusual bleeding or bruising, skin rashes, sedation, CNS depression, or respiratory depression.

### EXAMPLE 1

### Promethazine Hydrochloride, Hydrocodone Bitartrate and Acetaminophen Modified-Release Tablet

TABLE 1

| | | |
|---|---|---|
| Sr. | | Quantity/tablet |
| No. | Ingredients | (mg) |

Intra-granuler (Drug Layering)

| | | |
|---|---|---|
| 1 | Hydrocodone or salts (e.g. Bitratrate, HCl) | 5 to 12 |
| 2 | Inert sphere (e.g. Sugar sphere) | 50 to 200 |
| 3 | Binder (e.g. Povidone K30) | 1 to 5 |
| 4 | Opadry Clear (Barrier coat) | 1 to 5 |
| 5 | Ethylcellulose Aq. Dispersion, NF | 2 to 10 |
| | (Aquacoat ® ECD-30) | |
| 6 | Triethyl Citrate | 1 to 5 |
| 7 | Hypromellose (Methocel E3 Premium LV) | 2 to 10 |
| 8 | Talc (Lo Micron 5) | 1 to 5 |

Intra-granuler (wet granulation Dry granulation)

| | | |
|---|---|---|
| 9 | Acetaminophen 90% | 350 to 370 |
| 10 | Ethylcellulose Aq. Dispersion, NF | 10 to 50 |
| | (Aquacoat ® ECD-30) | |
| 11 | Water | Q.S. |

Extra-granuler (Dry mix)

| | | |
|---|---|---|
| 12 | Hydrocodone or salts (e.g. Bitratrate, HCl) | 1 to 3 |
| 13 | Microcrystalline Cellulose | 10 to 100 |
| 14 | Croscarmellose sodium | 10 to 30 |
| 15 | Silicified Microcrystalline Cellulose | 100 to 200 |
| 16 | Promethazine HCl | 10 to 15 |
| 17 | Sodium stearyl fumarate | 5 to 25 |

Procedure: Hydrocodone was layered on the sugar spheres. The hydrocodone coated sugar spheres were then coated with Ethylcellulose (EC) and a pore former such as HPMC to control the rate of release of the hydrocodone. Acetaminophen was granulated with EC or Eudragit® RL 30 D polymers to control the rate of release of Acetaminophen. Some amount of the hydrocodone was added to extra-granular part with Promethazine for providing an initial drug release.

What is claimed is:

1. A solid oral pharmaceutical composition comprising promethazine, hydrocodone and acetaminophen, or a pharmaceutically acceptable salt thereof, wherein the composition comprises:
(a) a plurality of inert spheres coated with one or more layers of hydrocodone, or a pharmaceutically acceptable salt thereof, and one or more outer layers of release rate controlling excipients;
(b) a plurality of granules comprising acetaminophen, or a pharmaceutically acceptable salt thereof, and one or more release rate controlling excipients; and
(c) an extra-granular admixture comprising promethazine, hydrocodone, acetaminophen, or a pharmaceutically acceptable salt thereof, and one or more pharmaceutically acceptable excipients.

2. The composition of claim 1, wherein the inert spheres are selected from sugar spheres, non-pareil seeds, microcrystalline cellulose beads, or mixtures thereof.

3. The composition of claim 1, wherein the hydrocodone layer and release rate controlling excipient layer in (a) are separated by a barrier layer.

4. The composition of claim 1, wherein the hydrocodone layer in (a) further comprise one or more binders and plasticizers.

5. The composition of claim 1, wherein the release rate controlling excipient layer in (a) further comprise one or more binders, pore-forming agents, plasticizers or mixtures thereof.

6. The composition of claim 1, wherein the granules in (b) further comprise one or more diluents, binders, disintegrating agents, or mixtures thereof.

7. The composition of claim 1, wherein the pharmaceutically acceptable excipients in the extra-granular admixture (c) comprises one or more diluents, binders, disintegrating agents, lubricants, or mixtures thereof.

8. The composition of claim 1, wherein the components (a) and (b) are formulated for controlled release and component (c) is formulated for immediate release.

9. The composition of claim 1, wherein the extra-granular admixture comprises up to 40% of the total amount of each of hydrocodone and acetaminophen, or a pharmaceutically acceptable salt thereof in the composition.

10. The composition of claim 1, comprise the promethazine, or a pharmaceutically acceptable salt thereof, the acetaminophen, or a pharmaceutically acceptable salt thereof, and the hydrocodone, or a pharmaceutically acceptable salt thereof in a mass ratio of about (1 to 2):(40 to 45):(1 to 2).

11. The composition of claim 1, wherein the hydrocodone layer in (a) comprises about 5 mg to about 12 mg hydrocodone, or a pharmaceutically acceptable salt thereof.

12. The composition of claim 1, wherein the extra-granular admixture (c) comprises about 1 mg to about 3 mg hydrocodone, or a pharmaceutically acceptable salt thereof.

13. The composition of claim 1, in the form of a tablet, a capsule, a mini-tablet or a caplet.

14. The composition of claim 1, wherein release of promethazine, hydrocodone, acetaminophen, or a pharmaceutically acceptable salt thereof from the composition commences simultaneously.

15. The composition of claim 1, wherein up to about 70% of the hydrocodone, or a pharmaceutically acceptable salt thereof, is released in about 12 minutes following contact of the composition with a dissolution fluid as measured by a USP Apparatus 2 (Paddle Apparatus).

16. The composition of claim 1, wherein up to about 85% of the hydrocodone, or a pharmaceutically acceptable salt thereof, is released in about 60 minutes following contact of the composition with a dissolution fluid as measured by a USP Apparatus 2 (Paddle Apparatus).

17. The composition of claim 1, wherein up to about 90% of the promethazine, or a pharmaceutically acceptable salt thereof, is released in about 12 minutes following contact of the composition with a dissolution fluid as measured by a USP Apparatus 2 (Paddle Apparatus).

18. A method for treating moderate to severe acute pain while preventing opioid induced nausea and vomiting comprising administering the pharmaceutical composition of claim 1 to a subject in need thereof.

19. A tablet comprising:
(a) a plurality of inert spheres coated with one or more layers comprising hydrocodone, or a pharmaceutically acceptable salt thereof, hydroxylpropyl cellulose, and one or more outer layers comprising ethyl cellulose, hydroxypropyl methylcellulose, triethyl citrate and talc;
(b) a plurality of granules comprising acetaminophen, or a pharmaceutically acceptable salt thereof, a release rate controlling excipient selected from ethyl cellulose or polymers and copolymers based on methacrylates and methacrylic acid or mixtures thereof; and
(c) an extra-granular admixture comprising promethazine, hydrocodone, acetaminophen, or a pharmaceutically acceptable salt thereof, croscarmellose sodium, silicified microcrystalline cellulose and sodium stearyl fumarate.

20. The tablet of claim 19, wherein the tablet consists of the plurality of spheres, the plurality of granules and the extra-granular admixture.
